(19) United States
(12) Patent Application Publication
(10) Pub. No.: US 2021/0158890 A1
(43) Pub. Date: May 27, 2021

(54) LATENT VARIABLE MODELING TO SEPARATE PCR BIAS AND BINDING AFFINITY

(71) Applicant: X Development LLC, Mountain View, CA (US)

(72) Inventors: Ivan Grubisic, Oakland, CA (US); David Brookes, Oakland, CA (US)

(21) Appl. No.: 16/692,522

(22) Filed: Nov. 22, 2019

(51) Int. Cl. G16B 5/00; G16B 30/00

(52) U.S. Cl. CPC G16B 5/00; G16B 30/00

(57) ABSTRACT

The present disclosure relates to development of aptamers, and in particular to developing machine-learning models to describe characteristics of a given sequence for an aptamer and based on the characteristics find other sequences for aptamers not observed experimentally, and techniques for separating out sequences for aptamers that are present primarily due to PCR bias and/or binding affinity. Particularly, aspects of the present disclosure are directed to obtaining sequence data for an aptamer sequence that binds to a target, generating a binding affinity latent variable and a PCR bias latent variable based on the sequence data, generating a predicted count of the aptamer sequence based on the binding affinity latent variable and PCR bias latent variable, determining that the binding affinity latent variable is greater than the PCR bias latent variable, and in response to the determining, accepting the predicted count of the aptamer sequence.

### FIELD

The present disclosure relates to development of aptamers, and in particular to developing machine-learning models to describe characteristics of a given sequence for an aptamer and based on the characteristics find other sequences for aptamers not observed experimentally, and techniques for separating out sequences for aptamers that are present primarily due to PCR bias and/or binding affinity.

### BACKGROUND

Aptamers are short sequences of single-stranded oligonucleotides (e.g., anything that is characterized as a nucleic acid, including xenobases). The sugar backbone of the single-stranded oligonucleotides functions as the acid and the A, T, C, G refers to the base. An aptamer can involve modifications to either the acid or the base. Aptamers have been shown to selectively bind to specific targets (e.g., proteins, protein complexes, peptides, carbohydrates, inorganic molecules, organic molecules such as metabolites, cells, etc.) with high binding affinity. Further, aptamers can be highly specific, in that a given aptamer may exhibit high binding affinity for one target but low binding affinity for many other targets. Thus, aptamers can be used to (for example) bind to disease-signature targets to facilitate a diagnostic process, bind to a treatment target to effectively deliver a treatment, bind to target molecules within a mixture to facilitate purification, etc. However, the utility of an aptamer hinges on a degree to which it effectively binds to a target.

Frequently, an iterative experimental process (e.g., Systematic Evolution of Ligands by EXponential Enrichment (SELEX)) is used to identify aptamers that are selectively bound to target molecules with high affinity. In the iterative experimental process, a nucleic acid library of oligonucleotide strands (aptamers) is incubated with a target molecule. Then, the target-bound oligonucleotide strands are separated from the unbound strands and amplified via polymerase chain reaction (PCR) to seed a new pool of oligonucleotide strands. This selection process is continued for a number (e.g., 6-15) rounds with increasingly stringent conditions, which ensure that the oligonucleotide strands obtained have the highest affinity to the target molecule.

The nucleic acid library typically includes 10¹⁴-10¹⁵ random oligonucleotide strands (aptamers). However, there are approximately a septillion (10²⁴) different aptamers that could be considered. Exploring this full space of candidate aptamers is impractical. However, given that present-day experiments are now only a sliver of the full space, it is highly likely that optimal aptamer selection is not currently being achieved. This is particularly true when it is important to assess the degree to which aptamers bind with multiple different targets, as a fewer portion of aptamers will have the desired combination of binding affinities across the targets. Accordingly, while substantive studies on aptamers have progressed since the introduction of the SELEX process, it would take an enormous amount of resources and time to experimentally evaluate a septillion (10²⁴) different aptamers every time a new target is proposed. In particular, there is a need for improving upon current experimental limitations with scalable machine-learning modeling techniques to identify aptamers and derivatives thereof that selectively bind to target molecules with high affinity.

### SUMMARY

In various embodiments, a computer-implemented method is provided that includes obtaining sequence data for an aptamer sequence that binds to a target; generating, by a binding affinity latent variable model, a binding affinity latent variable based on the sequence data; generating, by a polymerase chase reaction (PCR) bias latent variable model, a PCR bias latent variable based on the sequence data; generating, by a counting model, a predicted count of the aptamer sequence based on the binding affinity latent variable and PCR bias latent variable; determining that the binding affinity latent variable is greater than the PCR bias latent variable; and in response to the determining that the binding affinity latent variable is greater than the PCR bias latent variable, accepting the predicted count of the aptamer sequence.

In some embodiments, the sequence data comprises: (i) initial sequence data comprising a representation of the aptamer sequence and an observed count of the aptamer sequence in an initial library after a first amplification via the PCR; and (ii) selection sequence data comprising the representation of the aptamer sequence and an observed count of the aptamer sequence in a selection library after a second amplification via the PCR.

In some embodiments, the binding affinity latent variable is generated based on the selection sequence data, and the PCR bias latent variable is generated based on the initial sequence data and the selection sequence data.

In some embodiments, the generating the predicted count includes enforcing a constraint on a relationship between the binding affinity latent variable, the PCR bias latent variable, and the predicted count of the aptamer sequence, and where the relationship states as the binding affinity latent variable or the PCR bias latent variable increases or decrease an equivalent change of increasing or decreasing will be observed in the predicted count.

In some embodiments, the generating the predicted count further includes: predicting a count for the initial library based on the PCR bias latent variable; predicting a count for each cycle of a selection protocol based on the binding affinity latent variable and the PCR bias latent variable; and combining the count for the initial library and the count for each cycle of a selection protocol as a linear combination.

In some embodiments, the count for the initial library is connected to the PCR bias latent variable via a first bijective function, and the count for each cycle of the selection protocol is connected to the PCR bias latent variable and the affinity binding latent variable via the first bijective function and a second bijective function.

In some embodiments, the method further comprises in response to accepting the predicted count of the aptamer sequence, generating, by a sequence prediction model, one or more sequences based on the aptamer sequence.

In some embodiments, the method further comprises determining that the binding affinity latent variable is not greater than the PCR bias latent variable, and in response to the determining that the binding affinity latent variable is not greater than the PCR bias latent variable, rejecting the predicted count of the aptamer sequence.

In some embodiments, a system is provided that includes one or more data processors and a non-transitory computer readable storage medium containing instructions which, when executed on the one or more data processors, cause the one or more data processors to perform part or all of one or more methods or processes disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present invention will be better understood in view of the following non-limiting figures, in which:

FIG. 1 shows a block diagram of a aptamer development platform according to various embodiments;

FIG. 2 shows a machine-learning modeling system for separating out sequences of aptamers that are present primarily due to PCR bias and/or binding affinity in accordance with various embodiments;

FIGS. 3A and 3B show a concatenation of techniques for predicting sequence counts based on a binding affinity latent variable and a PCR bias latent variable in accordance with various embodiments;

FIG. 4 shows an exemplary flow for separating out sequences for aptamers that are present primarily due to PCR bias and/or binding affinity in accordance with various embodiments; and

FIG. 5 shows an exemplary computing device in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

I. Introduction

Systematic evolution of ligands by exponential enrichment (SELEX) is a powerful in vitro selection process conventionally used to identify oligonucleotide sequences (aptamers) with desired properties (usually high affinity for a target) from randomized nucleic acid libraries. A general disadvantage of SELEX is that each phase of the cycle can be labor-intensive, resource and time consuming, and require special expertise. In addition to this general problem, many of the technical disadvantages of SELEX originate from its dependency on polymerase chain reaction (PCR) for amplifying select oligonucleotide sequences. For example, while a nucleic acid library may contain equal amounts of different oligonucleotide sequences (aptamers), each of these oligonucleotide sequences may not be amplified to the same extent during PCR, and this can result in an unequal distribution of products. This effect, called PCR bias, may be exaggerated over multiple rounds of amplification. In some instances, it is possible to overcome PCR bias by carefully optimizing PCR conditions (e.g., annealing temperature). However, these techniques are not always practical and may not scale well when trying to evaluate a septillion (10²⁴) different aptamers.

To address these limitations and problems, machine-learning techniques disclosed herein can be used to identify in silico derived oligonucleotide sequences from in vitro or experimentally derived oligonucleotide sequences filtered to minimize or eliminate oligonucleotide sequences that would have been present primarily due to PCR bias. The identified in silico derived oligonucleotide sequences can then be tested in vitro or experimentally to assess binding affinities with one or more particular targets. For example, xeno nucleic acids (XNA) aptamer sequences may be run in vitro through multiple cycles of binding and amplification during selection schemes. XNA aptamer sequences such as threose nucleic acids (TNA) are synthetic nucleic acid analogues that have a different sugar backbone than the natural nucleic acids DNA and RNA. XNA may be selected for the aptamer sequences as these polymers are not readily recognized and degraded by nucleases, and thus are well-suited for in vivo applications. The readout after each in vitro selection cycle is to count the number of instances that each XNA aptamer sequence appears in the pool of target-bound XNA aptamers. Two characteristics generally lead to the presence of XNA aptamer sequences in the pool of target-bound XNA aptamers: PCR bias and binding affinity. For a machine-learning model to be useful at identifying particular aptamers for experiments, the primary driving factor that aptamer sequences are present in the pool of target-bound XNA aptamers should be binding affinity, and XNA aptamer sequences that are present in the pool of target-bound XNA aptamers primarily due to PCR bias should be separated or removed from the pool.

In order to determine whether XNA aptamer sequences are present in the pool of target-bound XNA aptamers primarily due to binding affinity or PCR bias, various embodiments are directed to machine-learning techniques for using latent variable models to infer PCR bias and binding affinity from other variables that are observed (directly measured or predicted). The dependencies for the machine-learning model include counting the XNA aptamer sequences present prior to any binding selection (e.g., the initial library), running in vitro at least one round of selection in the presence of a target, and then counting the XNA aptamer sequences present after binding selection (e.g., the pool of target-bound XNA aptamers). This means that a latent variable assigned to PCR bias acts on or impacts the count of aptamers in both the initial library and libraries created post each round of binding and selection (e.g., the pool of target-bound XNA aptamers). Whereas, a latent variable assigned to binding affinity acts only on or impacts the count of aptamers in the libraries created post each round of binding and selection (e.g., the pool of target-bound XNA aptamers). In the latent variable models, the latent variables are tied to the predicted counts in such a manner that increases/decreases in the latent variables will lead to the equivalent change in the counts of the XNA aptamer sequences present in the initial library and the pool of target-bound XNA aptamers. The net prediction of the counts is a linear combination of the latent variables and the previous round's count. This modeling strategy advantageously makes the latent variables humanly interpretable with respect to their individual quantities and influence on the overall modeling of selection schemes.

As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent. As used herein, when an action is “based on” something, this means the action is based at least in part on at least a part of the something.

It will be appreciated that techniques disclosed herein can be applied to assess other types of sequences rather than aptamers. It will also be appreciated that other types of latent variables are contemplated to infer other variables affecting sequence counts. For example, alternatively or additionally, a latent variable model may be used to model input library bias that favors the amplification of certain sequences over others.

II. Aptamer Development Techniques

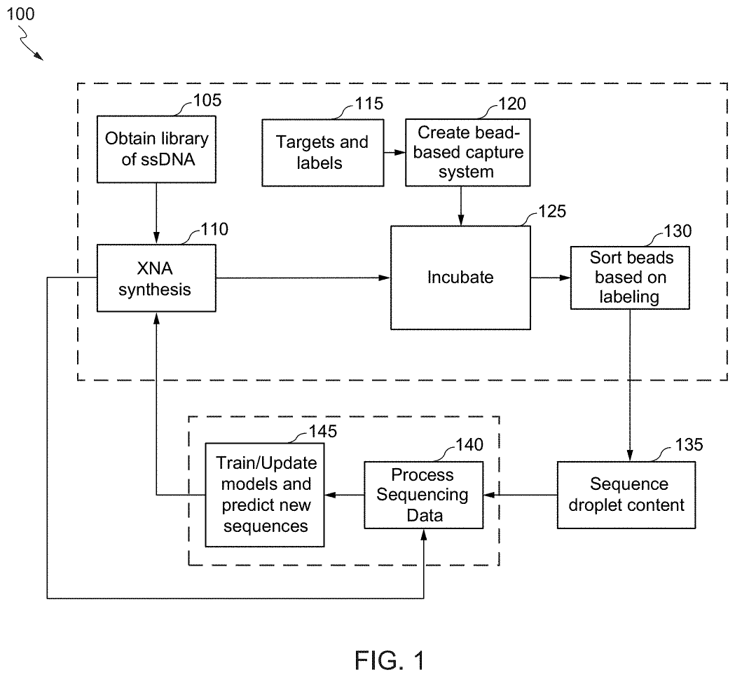

FIG. 1 shows a block diagram of an aptamer development platform 100 for strategically identifying particular aptamers for experiments to assess binding affinities with one or more particular targets. The aptamer development platform 100 includes obtaining one or more single stranded DNA or RNA (ssDNA or ssRNA) libraries at block 105. At block 110, the ssDNA or ssRNA are transcribed to synthesize a XNA aptamer library. For example, a TNA library of aptamers may be generated by primer extension of some or all of the oligonucleotide strands in a ssDNA library, flanking the aptamer sequences with fixed primer annealing sites for enzymatic amplification, and subsequent PCR amplification to create an XNA aptamer library that includes 10¹²-10¹⁵ aptamer sequences. The XNA aptamer library may be processed for application in downstream machine-learning processes. In some instances, the aptamer sequences are processed for use as training data, test data, or validation data in one or more machine-learning models. In other instances, the aptamer sequences are processed for use as actual experimental data in one or more trained machine-learning models. In either instance, the aptamer sequences may be processed to generate initial sequence data comprising a representation of the sequence of each aptamer and a count metric. The representation of the sequence can include one-hot encoding of each nucleotide in the sequence that maintains information about the order of the nucleotides in the aptamer. The representation of the sequence can additionally or alternatively include a string of category identifiers, with each category representing a particular nucleotide. The count metric can include a count of each aptamer in the library.

At block 115, a target (e.g., proteins, protein complexes, peptides, carbohydrates, inorganic molecules, organic molecules, cells, etc.) is obtained. In some instances the target is tagged with a label such as a fluorescent probe. At block 120, the labeled target is attached to beads to generate a bead-based capture system. In some instances, each bead is attached to a single labeled target molecule. The labeled target may be attached covalently to the beads, which may be polystyrene beads. At block 125, the bead-based capture system is incubated with the aptamers of the XNA aptamer library to allow for the aptamers to bind with the labeled target and form aptamer-target complexes.

At block 130, the beads having aptamer-target complexes are separated from the beads having non-binding sequences. At block 135, the aptamers from the aptamer-target complexes are eluted from the beads and target, and amplified by PCR to optionally prepare for subsequent rounds of selection (repeat blocks 110-130, for example a SELEX protocol). The stringency of the elution conditions can be increased to identify the tightest-binding or highest affinity sequences. Once the aptamers are separated and amplified, the aptamers may be sequenced to identify the sequence and count for each aptamer.

At block 140, the sequence and count for each aptamer that has gone through the selection process of steps 110-130 are processed for application in downstream machine-learning processes. In some instances, the sequence and count for each aptamer is processed for use as training data, test data, or validation data in one or more machine-learning models. In other instances, the sequence and count for each aptamer are processes for use as actual experimental data in one or more trained machine-learning models. In either instance, the sequence and count for each aptamer may be processed to generate selection sequence data comprising a representation of the sequence of each aptamer and a count metric. The representation of the sequence can include one-hot encoding of each nucleotide in the sequence that maintains information about the order of the nucleotides in the aptamer. The representation of the sequence can additionally or alternatively include other features concerning the sequence and/or aptamer, for example, post-translational modifications, binding sites, enzyme active sites, local secondary structure, kmers or characteristics identified for specific kmers, etc. The representation of the sequence can additionally or alternatively include a string of category identifiers, with each category representing a particular nucleotide. The count metric can include a count of the aptamer detected subsequent to an exposure to the target (e.g., during incubation and potentially in the presence of other aptamers). In some instances, the count metric can include a count of the aptamer detected subsequent to an exposure to the target in each round of selection.

At block 145, one or more machine-learning models are trained using the initial sequence data (from block 110) and the selection sequence data (from block 135). The one or more machine-learning models may include a neural network, such as a feedforward neural network, recurrent neural network, convolutional neural network, and/or a deep neural network. In various instances, the one or more machine-learning models include structures related to latent variables (e.g., the loss function and monotonic transformations) prior to training. The machine-learning models may be trained using training data, test data, and validation data based on sets of initial sequence data and selection sequence data to predict latent variables associated with binding affinity and PCR bias, predict counts of aptamer sequences, and predict sequences for derived aptamers (e.g., aptamers not experimentally determined by a selection process but predicted based on aptamers experimentally determined by a selection process). A loss function, such as an Mean Square Error (MSE) loss function, may be used to train each of the one or more machine-learning models. In some instances, a machine-learning model may be trained for the PCR bias using the initial sequence data and the selection sequence data. Another machine-learning model may be trained for the binding affinity using only the selection sequence data.

The trained machine-learning models can then be used to predict latent variables associated with binding affinity and PCR bias for the aptamers experimentally determined by the selection process (blocks 110-140), and predict counts based on the predicted latent variables associated with binding affinity and PCR bias for the aptamers experimentally. A subset of the aptamers experimentally determined by the selection process that have high predicted counts due primarily to high binding affinity (e.g., have a high associated binding affinity latent variable) can be identified and separated from aptamers experimentally determined by the selection process that have high predicted counts due primarily to PCR bias (e.g., have a high associated PCR bias latent variable). The subset of the aptamers experimentally determined by the selection process that have high predicted counts due primarily to high binding affinity can then be input into one or more machine learning models to identify in silico derived aptamer sequences (e.g., aptamer sequences that are derivatives of the experimentally selected aptamers).

The output can trigger experimental testing of some or all of the in silico derived aptamer sequences to experimentally measure binding affinities with the target and/or binding affinities with one or more other targets. The experimental testing may be conditioned on input from a client. For example, client device may present an interface in which the in silico derived aptamer sequences are identified along with input components configured to receive input to modify the in silico derived aptamer sequences (e.g., by removing or adding aptamers) and/or to generate an experiment-instruction communication to be sent to another device and/or other system. The experiment can include producing each of the in silico derived aptamer sequences. These aptamers can then be validated in the wet lab in either individual or bulk experiments. For example, the client can access a single aptamer (e.g. oligonucleotide). The single aptamer can be provided by an aptamer source, such as Twist Biosciences, Agilent, IDT, etc. The aptamer can be used to conduct biochemical assays (e.g. gel shift, surface plasma resonance, bio-layer interferometry, etc.). In some instances, multiple aptamers in a singular pool can be used to rerun the equivalent SELEX protocol (e.g., blocks 115-140) to identify enriched aptamers. Results can be assessed to determine whether the computational experiments are verified. In some instances, selections can be run in a digital format (i.e., ones that gave a functional output per sequence) to validate particular sequences. The validated sequences can be used to update the training set because the pair of sequence and affinity metric can be both normalized and calibrated.

III. Latent Variable Modeling Techniques to Separate PCR Bias and Binding Affinity

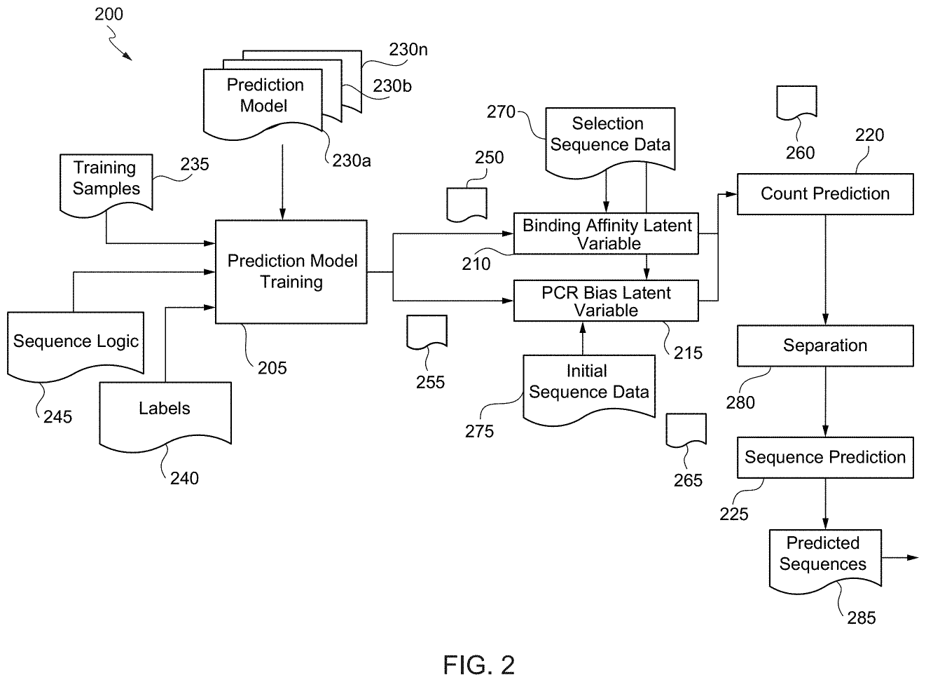

FIG. 2 shows a block diagram illustrating aspects of a machine-learning modeling system 200 for separating out sequences of aptamers that are present primarily due to PCR bias and/or binding affinity. As shown in FIG. 2, the predictions performed by the machine-learning modeling system 200 in this example include several stages: a prediction model training stage 205, a binding affinity latent variable prediction stage 210, a PCR bias latent variable prediction stage 215, a count prediction stage 220, and an aptamer prediction stage 225. The prediction model training stage 205 builds and trains one or more prediction models 230a-230n (‘n’ represents any natural number) to be used by the other stages (which may be referred to herein individually as a prediction model 230 or collectively as the prediction models 230). For example, the prediction models 230 can include a model for predicting latent variables associated with binding affinity in a constrained environment. The prediction models 230 can also include a model for predicting latent variables associated with PCR bias in a constrained environment. The prediction models 230 can also include a model for predicting counts of aptamer sequences based on the predicted binding affinity and PCR bias. The prediction models 350 can also include a model for predicting aptamer sequences. Still other types of prediction models may be implemented in other examples according to this disclosure.

A prediction model 230 can be a machine-learning model, such as a neural network, a convolutional neural network (“CNN”), e.g. an inception neural network, a residual neural network (“Resnet”) or NASNET provided by GOOGLE LLC from MOUNTAIN VIEW, Calif., or a recurrent neural network, e.g., long short-term memory (“LSTM”) models or gated recurrent units (“GRUs”) models. A prediction model 350 can also be any other suitable machine-learning model trained to predict latent variables, sequence counts or aptamer sequences from experimentally determined aptamer sequences, such as a support vector machine, decision tree, a three-dimensional CNN (“3DCNN”), a dynamic time warping (“DTW”) technique, a hidden Markov model (“HMM”), etc., or combinations of one or more of such techniques—e.g., CNN-HMM or MCNN (Multi-Scale Convolutional Neural Network). In various instances, at least one of the prediction models 230a-n includes structures related to latent variables (e.g., the loss function and monotonic transformations) prior to training. The machine-learning modeling system 200 may employ the same type of prediction model or different types of prediction models for latent variable, sequence count, and aptamer sequence prediction.

To train the various prediction models 230 in this example, training samples 235 for each prediction model 230 are obtained or generated. The training samples 235 for a specific prediction model 230 can include the initial sequence data and the selection sequence data as described with respect to FIG. 1 and optional labels 240 corresponding to the initial sequence data and the selection sequence data. For example, for a prediction model 230 to be utilized to predict a PCR bias latent variable for an aptamer sequence, the input can include the sequence and count features extracted from the initial sequence data and the selection sequence data associated with the sequence, and the optional labels 240 can include PCR features indicating parameters for the PCR or a vector indicating probabilities the initial sequence data and the selection sequence data include PCR bias. Similarly, for a prediction model 230 to be utilized to predict derived aptamer sequences based on a given sequence, the input can be the aptamer sequence itself or features extracted from the selection sequence data associated with the aptamer sequence and optional labels 240 can include known derivative sequences.

In some instances, the training process includes iterative operations to find a set of parameters for the prediction model 230 that minimizes a loss function for the prediction models 230. Each iteration can involve finding a set of parameters for the prediction model 230 so that the value of the loss function using the set of parameters is smaller than the value of the loss function using another set of parameters in a previous iteration. The loss function can be constructed to measure the difference between the outputs predicted using the prediction models 230 and the optional labels 240 contained in the training samples 235. Once the set of parameters are identified, the prediction model 230 has been trained and can be tested, validated, and/or utilized for prediction as designed.

In addition to the training samples 235, other auxiliary information can also be employed to refine the training process of the prediction models 230. For example, sequence logic 245 can be incorporated into the prediction model training stage 205 to ensure that the latent variables, counts, and aptamer sequences predicted by a prediction model 230 do not violate the sequence logic 245. For example, binding affinity (the strength of the binding interaction between an aptamer and a target) is a characteristic that can drive aptamers to be present in greater numbers in a pool of aptamer-target complexes after a cycle of selection process. Whereas PCR bias is a characteristic that can drive aptamers to be present in greater numbers in the initial library and/or a pool of aptamer-target complexes after a cycle of selection process. These relationships can be expressed in the sequence logic 245 such that as the binding affinity latent variable increases the predictive count increases (to represent this characteristic), as the binding affinity latent variable decreases the predictive count decreases, as the PCR bias latent variable increases the predictive count increases (to represent this characteristic), and as the PCR bias latent variable decrease the predictive count decreases. Moreover, an aptamer sequence generally has inherent logic among the different nucleotides. For example, GC content for an aptamer is typically not greater than 60%. This inherent logical relationship between GC content and aptamer sequences can be exploited to facilitate the aptamer sequence prediction.

According to some aspects of the disclosure presented herein, the logical relationship between the binding affinity and PCR bias can be formulated as one or more constraints to the optimization problem for training the prediction models 230. A training loss function that penalizes the violation of the constraints can be built so that the training can take into account the binding affinity and PCR bias constraints. Alternatively, or additionally, structures, such as a directed graph, that describe the current features and the temporal dependencies of the prediction output can be used to adjust or refine the features and predictions of the prediction models 230. In an example implementation, features may be extracted from the initial sequence data and combined with features from the selection sequence data as indicated in the directed graph. Features generated in this way can inherently incorporate the temporal, and thus the logical, relationship between the initial library and subsequent pools of aptamer sequences after cycles of the selection process. Accordingly, the prediction models 230 trained using these features can capture the logical relationships between sequence characteristics, selection cycles, aptamer sequences, and nucleotides.

Although the training mechanisms described herein mainly focus on training a prediction model 230, these training mechanisms can also be utilized to fine tune existing prediction models 230 trained from other datasets. For example, in some cases, a prediction model 230 might have been pre-trained using pre-existing aptamer sequence libraries. In those cases, the prediction models 230 can be retrained using the training samples 235 containing initial sequence data, experimentally derived selection sequence data, and other auxiliary information as discussed herein.

The prediction model training stage 205 outputs trained prediction models 230 including the trained binding affinity latent variable models 250, trained PCR bias latent variable models 255, trained count prediction models 260, and trained sequence models 265. The trained binding affinity latent variable models 250 may be used in the binding affinity latent variable stage 210 to generate binding affinity latent variable predictions based on selection sequence data 270. The trained PCR bias latent variable models 255 may be used in the PCR bias latent variable stage 215 to generate PCR bias latent variable predictions based on initial sequence data 275 and selection sequence data 270. The trained count prediction models 260 may be used in the count prediction stage 220 to generate count predictions based on the binding affinity latent variable predictions and the PCR bias latent variable predictions. The trained sequence models 265 may be used in the sequence prediction stage 225 to generate sequence predictions 285 for a subset of the selection sequence data 270 identified at separation stage 280 based on the binding affinity latent variable predictions, PCR bias latent variable predictions, and count predictions for the selection sequence data 270. In some instances, the separation stage 280 may separate the selection sequence data 270 into a first subset of sequences that have high predicted counts due primarily to high binding affinity (e.g., have a high associated binding affinity latent variable) and a second subset of sequences that have high predicted counts due primarily to PCR bias (e.g., have a high associated PCR bias latent variable). Some or all of the sequences in the first subset of sequences that have high predicted counts due primarily to high binding affinity can then be input into the trained sequence models 265 to identify sequence predictions 285 (i.e., in silico derived aptamer sequences that are derivatives of experimentally selected aptamers).

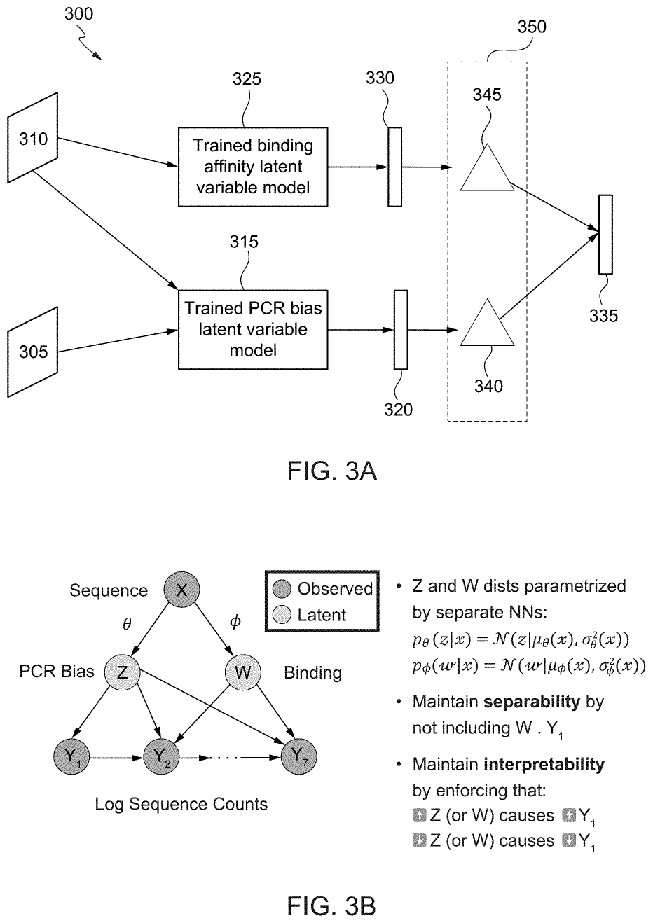

FIGS. 3A and 3B illustrate a concatenation of techniques 300 for predicting sequence counts based on a binding affinity latent variable and a PCR bias latent variable. In FIG. 3A, initial sequence data 305 from aptamer sequences prior to any binding and selection processes (e.g., the initial sequence data comprising a representation of the sequence of each aptamer and a count metric as described with respect to FIG. 1) and selection sequence data 310 from aptamer sequences after at least one binding and selection cycle (e.g., the selection sequence data comprising a representation of the sequence of each aptamer and a count metric as described with respect to FIG. 1) are input into a PCR bias latent variable model 315. The PCR bias latent variable model 315 (discussed in detail with respect to FIG. 3B) predicts a PCR bias latent variable 320 (a measure of the propensity of PCR bias to increase sequence counts) for a given aptamer sequence. In some instances, the PCR bias latent variable model 315 is implemented as a neural network model such as a feedforward neural network, recurrent neural network, convolutional neural network, and/or a deep neural network that relates observable variables within the initial sequence and count data 305 and selection sequence and count data 310 to a PCR bias latent variable 320.

The selection sequence data 310 from aptamer sequences after at least one binding and selection cycle (e.g., the selection sequence data comprising a representation of the sequence of each aptamer and a count metric as described with respect to FIG. 1) are input into an affinity binding latent variable model 325 (discussed in detail with respect to FIG. 3B) that predicts a binding affinity latent variable 330 (a measure of the propensity of binding affinity to increase sequence counts) for the given sequence. In some instances, the binding affinity latent variable model 325 is implemented as a neural network model such as a feedforward neural network, recurrent neural network, convolutional neural network, and/or a deep neural network that relates observable variables within the selection sequence and count data 310 to a binding affinity latent variable 330.

The connections between the PCR bias latent variable 320, the binding affinity latent variable 330, and net predictive count 335 for a given aptamer are trained bijections 340, 345 of count model 350. A bijection, bijective function, or one-to-one correspondence is a function between the elements of two sets (i.e., a latent variable and a sequence count) where each element of the first set (latent variable for initial library or subsequent selection cycle) is paired with exactly one element (count for initial library or subsequent selection cycle) of the second set. The net predictive count 335 for a given aptamer is linked via the bijective functions 340, 345 to increase or decrease as the PCR bias latent variable 320 or binding affinity latent variable 330 increases or decreases. For example, the bijective function 340 enforces that as PCR bias latent variable 320 for a given aptamer increases so does the net predictive count 330 of the given aptamer and as PCR bias latent variable 320 for the given aptamer decreases so does the net predictive count 330 of the given aptamer. Moreover, the bijective function 345 enforces that as binding affinity latent variable 330 for a given aptamer increases so does net predictive count 330 of the given aptamer and as binding affinity latent variable 330 for a given aptamer decreases so does the net predictive count 330 of the given aptamer. The net predictive count 330 is a linear combination of the latent variables 320, 330, the predicted count for an initial selection cycle and the predicted counts for each subsequent selection cycle thereafter.

FIG. 3B illustrates the PCR bias and binding affinity latent variable models. As shown, the PCR bias latent variable (Z) may be linked via parameters θ to a given aptamer sequence having observable variable (X) as the aptamer sequence progresses from the initial library Y₁ through subsequent selection cycles Y₂-Yₙ. A monte-carlo expectation-maximization algorithm −N(Z|μ_{θ}(x), σ²_{θ}(x)) may be used for learning the latent variable model p_{θ}(z|x) (e.g., PCR bias latent variable model 315) with parameters θ and latent variable (Z), as shown in Equation (1):

p_{θ}(z|x)=N(Z|μ_{θ}(x), σ²_{θ}(x)) Equation (1)

Where the latent variable model p is a probability distribution over two sets of variables (Z) and (X), and the variable (X) for a given aptamer sequence is observable and the PCR bias latent variable (Z) is unobservable. The expectation-maximization algorithm is an iterative two-step strategy: given an estimate θₜ of the weights, compute p_{θ}(z|x) and use it to compute the expected log-likelihood values for latent variable (Z), then find a new estimate of estimate θₜ₊₁ by optimizing the resulting tractable objective. This process will eventually converge.

The binding affinity latent variable (W) may be linked via parameters Φ to a given aptamer sequence having observable variable (X) as the aptamer sequence progresses from through selection cycles Y₂-Yₙ. A monte-carlo expectation-maximization algorithm −N(W|μ_{Φ}(x), σ²_{Φ}(x)) may be used for learning the latent variable model p_{θ}(z|x) (e.g., binding affinity latent variable model 325) with parameters Φ and latent variable (W), as shown in Equation (2):

p_{Φ}(z|x)=N(W|μ_{Φ}(x), σ²_{Φ}(x)) Equation (2)

Where the latent variable model p is a probability distribution over two sets of variables (W) and (X), and the variable (X) for a given aptamer sequence is observable and the binding affinity latent variable (W) is unobservable. The expectation-maximization algorithm is an iterative two-step strategy: given an estimate Φₜ of the weights, compute p_{θ}(z|x) and use it to compute the expected log-likelihood values for latent variable (Z), then find a new estimate of estimate Φₜ₊₁ by optimizing the resulting tractable objective. This process will eventually converge.

Separability between the two latent variables (W) and (Z) is maintained by not including Y₁ (the count from the initial library) within the binding affinity latent variable (W). In other words, not including Y₁ (the count from the initial library) within the binding affinity latent variable (W) allows for the two latent variables to be differentiable from one another, and thus sequences that have a high count due primarily to PCR bias may be separated from sequences that have a high count primarily due to affinity bias in a downstream process. It will also be appreciated that other types of separability between latent variables are contemplated. For example, alternatively or additionally, other observed counts that only connect to latent variable (Z) (e.g., a measurement of sequence bias after a first round of selection) or only connect to latent variable (W) (e.g., a measurement of binding affinity after a first round of selection). The prediction of counts from each step Y₁-Yₙ is linked via bijective functions to the increase or decrease in accordance with latent variables (Z) and (W). For example, the interpretability of the predicted counts is maintained by enforcing that as PCR bias latent variable (Z) increases so does the count for the respective initial library Y₁ or subsequent selection cycles Y₂-Yₙ and as PCR bias latent variable 320 (Z) decreases so does the count for the respective initial library Y₁ or subsequent selection cycles Y₂-Yₙ. The interpretability of the predicted counts is further maintained by enforcing that as the binding affinity latent variable (W) increases so does the count for the respective selection cycles Y₂-Yₙ and as binding affinity latent variable (W) decreases so does the count for the respective selection cycles Y₂-Yₙ. The net predicted count associated with a given aptamer sequence is a linear combination with positive parameter of the individual counts for the initial library Y₁ and subsequent selection cycles Y₂-Yₙ.

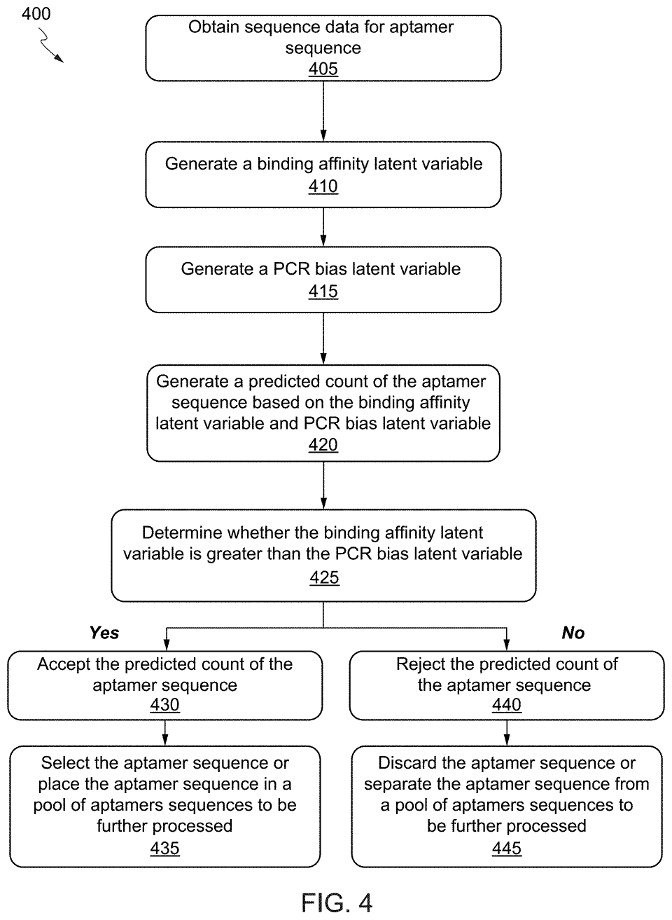

FIG. 4 is a simplified flow chart 400 illustrating an example of processing for separating out sequences for aptamers that are present primarily due to PCR bias and/or binding affinity using an aptamer development platform and a machine-learning modeling system and technique (e.g., the aptamer development platform 100 and machine-learning modeling system and technique 200, 300 described with respect to FIGS. 1, 2, 3A, and 3B). Process 400 begins at block 405, at which sequence data is obtained for an aptamer sequence that binds to a target. the sequence data comprises: (i) initial sequence data comprising a representation of the aptamer sequence and an observed count of the aptamer sequence in an initial library after a first amplification via the PCR; and (ii) selection sequence data comprising the representation of the aptamer sequence and an observed count of the aptamer sequence in a selection library after a second amplification via the PCR. At block 410, a binding affinity latent variable is generated based on the sequence data. The binding affinity latent variable may be generated using a binding affinity latent variable model, e.g., a feedforward neural network. In some instances, the binding affinity latent variable is generated based on the selection sequence data. At block 415, a PCR bias latent variable is generated based on the sequence data. The PCR bias latent variable may be generated using a PCR bias latent variable model, e.g., a feedforward neural network. In some instances, the PCR bias latent variable is generated based on the initial sequence data and the selection sequence data.

At block 420, a predicted count of the aptamer sequence is generated based on the binding affinity latent variable and PCR bias latent variable. In some instances, the generating the predicted count includes enforcing a constraint on a relationship between the binding affinity latent variable, the PCR bias latent variable, and the predicted count of the aptamer sequence. The relationship states as the binding affinity latent variable or the PCR bias latent variable increases or decrease an equivalent change of increasing or decreasing will be observed in the predicted count. In some instance, the generating the predicted count further includes predicting a count for the initial library based on the PCR bias latent variable, predicting a count for each cycle of a selection protocol based on the binding affinity latent variable and the PCR bias latent variable, and combining the count for the initial library and the count for each cycle of a selection protocol as a linear combination. The count for the initial library may be connected to the PCR bias latent variable via a first bijective function, and the count for each cycle of the selection protocol may be connected to the PCR bias latent variable and the affinity binding latent variable via the first bijective function and a second bijective function

At block 425, a determination is made as to whether the binding affinity latent variable is greater than the PCR bias latent variable. When the binding affinity latent variable is greater than the PCR bias latent variable, the process continues at block 430 where the predicted count of the aptamer sequence is accepted. At block 435, in response to accepting the predicted count of the aptamer sequence, the aptamer sequence is selected or placed in a pool of aptamers sequences to be further processed. The further processing may comprise generating one or more derivative sequences based on the aptamer sequence. The one or more derivative sequences may be generated using a sequence prediction model. When the binding affinity latent variable is not greater than the PCR bias latent variable (less than or equal to the PCR bias latent variable), the process continues at block 440 where the predicted count of the aptamer sequence is rejected. At block 445, in response to rejecting the predicted count of the aptamer sequence, the aptamer sequence is discarded or separated from a pool of aptamers sequences to be further processed.

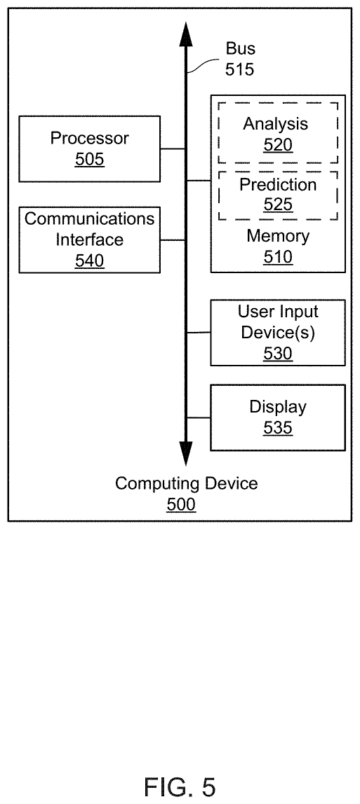

FIG. 5 illustrates an example computing device 500 suitable for use with systems and methods for separating out sequences for aptamers that are present primarily due to PCR bias and/or binding affinity according to this disclosure. The example computing device 500 includes a processor 505 which is in communication with the memory 510 and other components of the computing device 500 using one or more communications buses 515. The processor 505 is configured to execute processor-executable instructions stored in the memory 510 to perform one or more methods for separating out sequences for aptamers that are present primarily due to PCR bias and/or binding affinity according to different examples, such as part or all of the example method 400 described above with respect to FIG. 4. In this example, the memory 510 stores processor-executable instructions that provide sequence data analysis 520 and latent variable/sequence count prediction 525, as discussed above with respect to FIGS. 1, 2, 3A, 3B, and 4.

The computing device 500, in this example, also includes one or more user input devices 530, such as a keyboard, mouse, touchscreen, microphone, etc., to accept user input. The computing device 500 also includes a display 535 to provide visual output to a user such as a user interface. The computing device 500 also includes a communications interface 540. In some examples, the communications interface 540 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

IV. Additional Considerations

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A computer-implemented method comprising:
obtaining sequence data for an aptamer sequence that binds to a target;
generating, by a binding affinity latent variable model, a binding affinity latent variable based on the sequence data;
generating, by a polymerase chase reaction (PCR) bias latent variable model, a PCR bias latent variable based on the sequence data;
generating, by a counting model, a predicted count of the aptamer sequence based on the binding affinity latent variable and PCR bias latent variable;
determining that the binding affinity latent variable is greater than the PCR bias latent variable; and
in response to the determining that the binding affinity latent variable is greater than the PCR bias latent variable, accepting the predicted count of the aptamer sequence.

2. The method of claim 1, wherein the sequence data comprises: (i) initial sequence data comprising a representation of the aptamer sequence and an observed count of the aptamer sequence in an initial library after a first amplification via the PCR; and (ii) selection sequence data comprising the representation of the aptamer sequence and an observed count of the aptamer sequence in a selection library after a second amplification via the PCR.

3. The method of claim 2, wherein the binding affinity latent variable is generated based on the selection sequence data, and the PCR bias latent variable is generated based on the initial sequence data and the selection sequence data.

4. The method of claim 3, wherein the generating the predicted count includes enforcing a constraint on a relationship between the binding affinity latent variable, the PCR bias latent variable, and the predicted count of the aptamer sequence, and wherein the relationship states as the binding affinity latent variable or the PCR bias latent variable increases or decrease an equivalent change of increasing or decreasing will be observed in the predicted count.

5. The method of claim 4, wherein the generating the predicted count further includes:
predicting a count for the initial library based on the PCR bias latent variable;
predicting a count for each cycle of a selection protocol based on the binding affinity latent variable and the PCR bias latent variable; and
combining the count for the initial library and the count for each cycle of a selection protocol as a linear combination, and
wherein the count for the initial library is connected to the PCR bias latent variable via a first bijective function, and the count for each cycle of the selection protocol is connected to the PCR bias latent variable and the affinity binding latent variable via the first bijective function and a second bijective function.

6. The method of claim 1, further comprising in response to accepting the predicted count of the aptamer sequence, generating, by a sequence prediction model, one or more sequences based on the aptamer sequence.

7. The method of claim 1, further comprising:
determining that the binding affinity latent variable is not greater than the PCR bias latent variable; and
in response to the determining that the binding affinity latent variable is not greater than the PCR bias latent variable, rejecting the predicted count of the aptamer sequence.

8. A system comprising:
one or more data processors; and
a non-transitory computer readable storage medium containing instructions which, when executed on the one or more data processors, cause the one or more data processors to perform actions including:
obtaining sequence data for an aptamer sequence that binds to a target;
generating, by a binding affinity latent variable model, a binding affinity latent variable based on the sequence data;
generating, by a polymerase chase reaction (PCR) bias latent variable model, a PCR bias latent variable based on the sequence data;
generating, by a counting model, a predicted count of the aptamer sequence based on the binding affinity latent variable and PCR bias latent variable;
determining that the binding affinity latent variable is greater than the PCR bias latent variable; and
in response to the determining that the binding affinity latent variable is greater than the PCR bias latent variable, accepting the predicted count of the aptamer sequence.

9. The system of claim 8, wherein the sequence data comprises: (i) initial sequence data comprising a representation of the aptamer sequence and an observed count of the aptamer sequence in an initial library after a first amplification via the PCR; and (ii) selection sequence data comprising the representation of the aptamer sequence and an observed count of the aptamer sequence in a selection library after a second amplification via the PCR.

10. The system of claim 9, wherein the binding affinity latent variable is generated based on the selection sequence data, and the PCR bias latent variable is generated based on the initial sequence data and the selection sequence data.

11. The system of claim 10, wherein the generating the predicted count includes enforcing a constraint on a relationship between the binding affinity latent variable, the PCR bias latent variable, and the predicted count of the aptamer sequence, and wherein the relationship states as the binding affinity latent variable or the PCR bias latent variable increases or decrease an equivalent change of increasing or decreasing will be observed in the predicted count.

12. The system of claim 11, wherein the generating the predicted count further includes:
predicting a count for the initial library based on the PCR bias latent variable;
predicting a count for each cycle of a selection protocol based on the binding affinity latent variable and the PCR bias latent variable; and
combining the count for the initial library and the count for each cycle of a selection protocol as a linear combination, and
wherein the count for the initial library is connected to the PCR bias latent variable via a first bijective function, and the count for each cycle of the selection protocol is connected to the PCR bias latent variable and the affinity binding latent variable via the first bijective function and a second bijective function.

13. The method of claim 8, wherein the actions further include in response to accepting the predicted count of the aptamer sequence, generating, by a sequence prediction model, one or more sequences based on the aptamer sequence.

14. The method of claim 8, wherein the actions further include:
determining that the binding affinity latent variable is not greater than the PCR bias latent variable; and
in response to the determining that the binding affinity latent variable is not greater than the PCR bias latent variable, rejecting the predicted count of the aptamer sequence.

15. A computer-program product tangibly embodied in a non-transitory machine-readable storage medium, including instructions configured to cause one or more data processors to perform actions including:
obtaining sequence data for an aptamer sequence that binds to a target;
generating, by a binding affinity latent variable model, a binding affinity latent variable based on the sequence data;
generating, by a polymerase chase reaction (PCR) bias latent variable model, a PCR bias latent variable based on the sequence data;
generating, by a counting model, a predicted count of the aptamer sequence based on the binding affinity latent variable and PCR bias latent variable;
determining that the binding affinity latent variable is greater than the PCR bias latent variable; and
in response to the determining that the binding affinity latent variable is greater than the PCR bias latent variable, accepting the predicted count of the aptamer sequence.

16. The computer-program product of claim 15, wherein the sequence data comprises: (i) initial sequence data comprising a representation of the aptamer sequence and an observed count of the aptamer sequence in an initial library after a first amplification via the PCR; and (ii) selection sequence data comprising the representation of the aptamer sequence and an observed count of the aptamer sequence in a selection library after a second amplification via the PCR.

17. The computer-program product of claim 16, wherein the binding affinity latent variable is generated based on the selection sequence data, and the PCR bias latent variable is generated based on the initial sequence data and the selection sequence data.

18. The computer-program product of claim 17, wherein the generating the predicted count includes enforcing a constraint on a relationship between the binding affinity latent variable, the PCR bias latent variable, and the predicted count of the aptamer sequence, and wherein the relationship states as the binding affinity latent variable or the PCR bias latent variable increases or decrease an equivalent change of increasing or decreasing will be observed in the predicted count.

19. The computer-program product of claim 18, wherein the generating the predicted count further includes:
predicting a count for the initial library based on the PCR bias latent variable;
predicting a count for each cycle of a selection protocol based on the binding affinity latent variable and the PCR bias latent variable; and
combining the count for the initial library and the count for each cycle of a selection protocol as a linear combination, and
wherein the count for the initial library is connected to the PCR bias latent variable via a first bijective function, and the count for each cycle of the selection protocol is connected to the PCR bias latent variable and the affinity binding latent variable via the first bijective function and a second bijective function.

20. The computer-program product of claim 15, wherein the actions further include:
determining that the binding affinity latent variable is not greater than the PCR bias latent variable; and
in response to the determining that the binding affinity latent variable is not greater than the PCR bias latent variable, rejecting the predicted count of the aptamer sequence.
